SEQUENCE LISTING (8 sequences)

SEQ ID NO: 1 - DNA, 20 bp, Artificial
  primer
aggctcaaggaaaaacatgg

SEQ ID NO: 2 - DNA, 20 bp, Artificial
  primer
gcagaatgcccagagtcttc

SEQ ID NO: 3 - DNA, 20 bp, Artificial
  primer
gcagctgagcttgcagaata

SEQ ID NO: 4 - DNA, 25 bp, Artificial
  primer
ggacaaagtataggagaaacaacca

SEQ ID NO: 5 - DNA, 20 bp, Artificial
  primer
agcatcgggtgatgttcatt

SEQ ID NO: 6 - DNA, 20 bp, Artificial
  primer
attacaagcatgcgtcacca

SEQ ID NO: 7 - DNA, 20 bp, Artificial
  primer
tggagggtgtccgcaatgtt

SEQ ID NO: 8 - DNA, 20 bp, Artificial
  primer
gaaggccttgaccttttcag

(19) United States
(12) Patent Application Publication
(10) Pub. No.: US 2011/0190220 A1
(43) Pub. Date: Aug. 4, 2011

(54) Use of Defensin Alpha 1 and/or Defensin Alpha 4, as a Marker for Predicting Treatment Response and/or a Relapse in a Patient Suffering form Chronic Myeloid Leukemia

(76) Inventors: Béatrice Turcq, Bordeaux (FR); Gabriel Etienne, Bordeaux (FR); Maryse Dupouy, Pessac (FR); François-Xavier Mahon, Bordeaux (FR); Bertrand Garbay, Gradignan (FR); Patricia Costaglioli, Bordeaux (FR)

(21) Appl. No.: 13/054,034

(22) PCT Filed: Jul. 17, 2009
(86) PCT No.: PCT/IB2009/006622

§ 371 Date: Mar. 10, 2011

(30) Foreign Application Priority Data
Jul. 18, 2008 (IB) PCT/IB2008/002795

(51) Int. Cl. A61K 38/02; C12Q 1/68; G01N 33/566; A61K 31/506; A61K 35/02

(52) U.S. Cl. 514/19.6; 435/6.13; 436/501; 435/6.11; 435/6.12; 514/252.18; 514/275; 514/252.19

(57) ABSTRACT

The present invention pertains to the use of defensin α1 and/or defensin α4, as a marker for predicting and following the response of a patient suffering from chronic myeloid leukaemia to a treatment with imatinib.

Chronic Myeloid Leukemia (CML) is a clonal hematologic malignant disease of the hematopoietic stem cell in which a t(9; 22) (q34; q11) reciprocal translocation leads to a 22 abnormal chromosome designated Philadelphia chromosome (Ph). Translocation fuses BCR and ABL genetic sequences resulting in the BCR-ABL hybrid gene coding the fusion protein BCR-ABL (Faderl et al., 1999). This constitutively activated tyrosine kinase protein is responsible of the transformation of BCR-ABL cells.

Recently, novel therapeutic molecules have been used to treat CML patients. Imatinib mesylate (Glivec, or Gleevec in the US; Novartis, Switzerland), hereafter referred to as “imatinib”, is a tyrosine kinase inhibitor that blocks the BCR-ABL kinase activity. Imatinib treatment inhibits specifically leukemia cells of CML patients. This molecule inhibits deregulated BCR-ABL protein tyrosine kinase activity and selectively eradicates CML cells. Imatinib is the major therapeutic agent for the treatment of patients with CML.

Imatinib can induce a complete or nearly complete cytogenetic remission in up to 80 percent of patients when they are treated in chronic phase (Deininger et al., 2005).

However, in 20 percent of CML patients, this treatment is inefficient. Indeed, about 20% of CML patients treated with imatinib are or become resistant to treatment. Two kinds of resistances can be distinguished:
in primary resistance, patients never respond to treatment;
in secondary resistance, patients first respond to treatment, but after a few months or years, they escape.

In some cases, mutation in the BCR-ABL tyrosine kinase domain can explain a newly occurring resistance. However, in other cases, no mutation allows to explain the resistance phenomenon.

In this context, there is a strong need for molecular markers which could help physicians to predict the response of a given patient to imatinib, and/or to detect a secondary resistance at a very early stage, so that the treatment of said patient can be adapted.

To this aim, and as described in the experimental part below, the inventors have used a pangenomic microarray to analyse a great number of genes in CML patients in different situations (chronic disease before imatinib treatment, during a successful treatment, after relapse due to a secondary resistance, etc.). By doing so, they have identified two prognosis markers for the response to imatinib and for secondary resistance to this treatment. These markers are defensin α1 and defensin α4.

Defensins are small antimicrobial peptides involved in innate and adaptive defense systems. By their antibacterial activities, they permeabilize bacterial membrane. Defensins α1 and α4 are synthesized as preprodefensins in the bone marrow precursors of blood granulocytes. The mature defensins are then stored in the granules of neutrophils.

Interestingly, in a transcriptomic study for identifying markers associated with the progression of chronic myeloid leukaemia and resistance to imatinib, Radich et al. failed to identify these defensins as markers for determining a patient's response to imatinib (US 2007/0154931).

In another transcriptomic study, Nakamura and Katagiri demonstrated that defensin α1 is over-expressed in CML patients (US 2007/0092519). However, these authors did not try to establish a correlation of the expression level of this defensin with patients' response to imatinib treatment.

The present invention hence pertains to the use of a defensin selected in the group consisting of defensin α1, defensin α2, defensin α3, and defensin α4, or of a combination thereof, as a marker for predicting the response of a patient suffering from chronic myeloid leukaemia to a treatment with a tyrosine kinase inhibitor. In a preferred embodiment, defensin α1 and/or defensin α4 are used according to the invention. Indeed, as explained in Example 6 below, no gene for defensin α2 has been discovered (this defensin is probably a proteolytic product of defensin α1 and/or defensin α3), and defensin α3 is not present in all individuals. However, due to the small extent of the difference between defensin a 1, defensin α2 and defensin α3, most primers designed to measure the level of defensin α1 mRNA also amplify defensin α3 mRNA. In what follows, the phrase “expression level of defensin al” will designate either the expression level of this sole defensin, or the expression level of the locus DEFA1A3.

In particular, defensin α1 and/or defensin α4 can be used as a marker for predicting the response of a patient suffering from chronic myeloid leukaemia to a treatment with tyrosine kinase inhibitors such as imatinib, nilotinib or dasatinib.

The present invention also relates to a method for predicting relapse in a patient who is treated or who has been treated for a chronic myeloid leukaemia, wherein said method comprises the following steps:

a) in vitro measuring the expression level of defensin α1 and/or defensin α4;

b) comparing the measured expression level of defensin α1 and/or defensin α4 to a predetermined threshold;

wherein a measured level above said predetermined threshold is indicative of relapse.

In the above method, the in vitro measure can be performed on various kinds of biological samples from said patient, such as blood, serum, saliva or urine samples.

The physician can determine the appropriate threshold to perform the above method by different calculation methods. The physician will chose the calculation method depending on the context.

In a preferred embodiment, the physician will use the patient as his/her own reference, by calculating the predetermined threshold as 10×P, wherein P is the level of the same marker (defensin α1 and/or defensin α4) previously measured in the same patient, when said patient's condition was improving or stabilized. Of course, this can be done only when a “reference biological sample” from this patient is available, i.e., when a biological sample was obtained at a time when the patient's condition was improving or stabilized, which means, in the present context, that the level of BCR-ABL in said patient was decreasing or not detectable.

When no “reference biological sample” from said patient is available, the threshold must be determined by reference to values generally observed in a cohort of patients. As appears from the experimental part below, when the defensin α1 expression level is calculated as a ratio between defensin α1 mRNA copy number and 13-actin mRNA copy number (measured by quantitative PCR), the value of 10⁴ can be taken as predetermined threshold. An appropriate predetermined threshold for defensin α4 is 10, if the expression level is calculated as a ratio between defensin α4 mRNA copy number and β-actin mRNA copy number (measured by quantitative PCR). Of course, the skilled artisan can easily determine the equivalent value when a different technique is used to measure the expression level of defensin α1 and/or defensin α4, for example if a gene different from β-actin is used as a reference, of if the expression level is measured at the protein level instead of the mRNA level. By reproducing the inventors' experiments on a larger cohort of patients, the skilled artisan can also determine more precisely a relevant threshold, and calculate the significant statistical parameters (such as sensibility and specificity) corresponding to this threshold.

Another aspect of the present invention is a method for following the evolution of a CML patient, still under treatment and/or under full remission, by regularly measuring the expression level of defensin α1 and/or defensin α4 in said patient. According to this embodiment, the method comprises the following steps:

a) in vitro measuring the expression level of defensin α1 and/or defensin α4 in biological samples from said patient, wherein said biological samples have been obtained at various dates;

b) comparing the measured expression levels of defensin α1 and/or defensin α4 in said biological samples;

wherein an increase in the level of expression of defensin α1 and/or defensin α4 in said patient is indicative of relapse.

In this method, biological samples from said patient will be regularly obtained. Advantageously, the expression level of defensin α1 and/or defensin α4 will be measured every one to three month(s) during the treatment of the disease. This rhythm can be slowed once the patient is in complete remission and still under treatment. After the arrest of imatinib treatment in case of complete remission, the physician can chose to measure the level of defensin α1 and/or defensin α4 every month during at least 6 months, in order to quickly detect a relapse. Of course, the physician will adapt the frequency of the measures according to the evolution of each patient.

As mentioned, the above methods can be performed for anticipating a relapse in a patient still under treatment, for example for a patient treated with imatinib, nilotinib or dasatinib. In this case, an increase in the expression level of defensin al and/or defensin α4 indicates that the patient's treatment must be changed in order to avoid relapse. Indeed, this can correspond to a secondary resistance to the inhibitor, necessitating an increase of the dose of drug given to said patient, or a switch to another tyrosine kinase inhibitor, or a bone marrow graft. The above methods can also be used for detecting relapse in a person who has been treated for a chronic myeloid leukaemia but who is considered in complete remission. Indeed, in case of relapse, the defensin al and/or defensin α4 expression level will increase a few weeks, possibly a few months before BCR-ABL becomes detectable in said patient. The use of a very early marker of relapse such as those according to the present invention can hence prevent a degradation of the subject's condition, by treating the person again before the appearance of the clinical symptoms.

As disclosed in Examples 5 and 8 below, the inventors have also shown, in a retrospective study involving CML patients responding to imatinib and CML patients who were not good responders to this treatment, that the level of defensins at the beginning of the treatment was statistically superior in patients who are good responders to imatinib. The same results have been observed (data not shown) with other tyrosine kinase inhibitors, in particular with nilotinib and dasatinib.

Hence, according to yet another aspect, the present invention concerns a method for determining if a patient suffering from chronic myeloid leukaemia is likely to be a good responder to a treatment with a tyrosine kinase inhibitor, comprising the following steps:

a) in vitro measuring the expression level of defensin α1 and/or defensin α4;

b) comparing the measured expression level of defensin α1 and/or defensin α4 to a predetermined threshold;

wherein a measured level above said predetermined threshold indicates that said patient is likely to be a good responder to the treatment.

Of course, the predetermined threshold will be calculated, depending on the technology used to measure the level of defensin α1 and/or defensin α4, by performing a statistical study on a representative cohort. A “representative cohort” is a cohort of patients who have undergone a chronic myeloid leukaemia and have been treated with imatinib, and for whom the response to the considered tyrosine kinase inhibitor is known. A representative cohort must comprise at least 35 patients, with at least 10 in each group (responders vs/non responders). Of course, the skilled artisan can chose to use a bigger cohort for implementing the present invention, and the members of the representative cohort must have been chosen without any selection bias. For example, when defensin a 1 expression is calculated as the log of the ratio [defensin α1 mRNA level/β-actin mRNA level], it can be considered that a patient having a defensin α1 expression level above 5 is likely to be a good responder to imatinib.

It is important to early identify patients likely to be primary or secondary resistant to a tyrosine kinase inhibitor such as imatinib, since an adapted treatment can be proposed. In particular, these patients can be considered as having priority for a bone marrow graft.

The inventors have also demonstrated (Example 9 below) that the level of expression of defensin α1 and/or defensin α4 can be used to predict if a patient under unsatisfactory treatment with a tyrosine kinase inhibitor will be a good responder to an increased dose of the same tyrosine kinase inhibitor. The present invention hence also pertains to a method for predicting if a patient suffering from chronic myeloid leukaemia and for whom a treatment with a tyrosine kinase inhibitor is insufficient can benefit from an increase of daily dose of said tyrosine kinase inhibitor, comprising the following steps:

a) in vitro measuring the expression level of defensin α1 and/or defensin α4 prior to and after the dose increase;

b) comparing the measured expression levels of defensin α1 and/or defensin α4;

wherein a decrease of al and/or defensin α4 expression level following the dose increase is indicative of a good response, whereas an increase of said expression level is indicative of treatment failure.

When performing the above method, a treatment is considered as “insufficient” if an increase of BCR-ABL and/or defensin α1 and/or defensin α4 has been observed under this treatment. The measure of the level of defensin α1 and/or defensin α4 prior to the dose increase can be performed up to the day of said dose increase, whereas the measure after the dose increase will preferably be performed at least one week after the dose increase, more preferably a few weeks or a few months after the dose increase, so that the effect is apparent.

According to a preferred embodiment of the above methods, said inhibitor is imatinib, nilotinib or dasatinib.

When performing the above methods, the skilled artisan can chose to measure either the expression level of defensin α1, or the expression level of defensin α4, or both of them. As explained above, except if specific primers are used to discriminate defensin α1 from defensin α3, the measure of defensin α1 mRNA by RT-PCR is representative of the expression level of the whole DEFA1A3 locus. Of course, the physician can combine these markers to other biological markers of the disease, for example the level of BCR-ABL.

A particular technique to measure the expression level of defensin al and/or defensin α4, illustrated in the examples below, is quantitative polymerase chain reaction after a reverse transcription step. Examples of primers which can be used for this reaction are disclosed in the experimental part below. As also disclosed below, the skilled artisan can chose to calculate the expression level of defensin α1 and/or defensin α4 is as a ratio between the copy number of mRNA encoding said defensin and the copy number of mRNA encoding a reference gene. By “reference gene” is herein meant a gene which has a stable expression. Examples of such a gene are β actin, HuP0, Gus, ABL, . . . .

Of course, any other technique can be used to measure the expression level of defensin α1 and/or defensin α4 in biological samples. For example, this expression level can be measured at the protein level instead of the mRNA level as mentioned above, for example by using an immunoassay. Indeed, antibodies targeting defensin α1 and/or defensin α4 are already available, such as the D21 monoclonal antibody sold by HyCult biotechnology b. v. (The Netherlands) under the references HM2058 and HM2059. HyCult biotechnology b. v. also provides an ELISA kit for detecting human neutrophil defensins, under the references HK314, HK315, HK317, HK321, HK324 and HK325. Such a kit can be used to perform the present invention.

Since the inventors have demonstrated that CML patients having a higher level of defensin α1 and/or defensin α4 at the beginning of treatment with a tyrosine kinase inhibitor such as imatinib, nilotinib and dasatinib respond to this treatment better than those who have a lower level of defensins, they hypothesized that defensin α1 and/or defensin α4 are involved in the immune response against cancer, at least in chronic myeloid leukaemia, and that administration of defensins (α1, α2, α3 and/or α4) to the patients, alone or combined to a tyrosine kinase inhibitor, can improve their status. Alternatively, agents causing an increase of defensins expression in patients can be administered instead of defensins themselves. Such agents can be, for example, bacteria or fungi, since defensins are secreted in the primary response to such micro-organisms. Accordingly, the present invention also pertains to the use of defensin α1, α2, α3 and/or α4, and/or agents inducing an increase of defensins in humans, for the preparation of a drug for: (i) treating cancer and preventing relapse, in particular in the case of hematologic cancers such as chronic myeloid leukaemia, and/or (ii) increasing the efficiency of imatinib, nilotinib and dasatinib and other tyrosine kinase inhibitors in the treatment of CML.

When used in combination with a tyrosine kinase inhibitor such as imatinib, nilotinib or dasatinib defensins, and/or the above-described agent can be administered either at the same time as said inhibitor, or separately (before of after). Accordingly, the present invention pertains to a medicinal product comprising (i) a tyrosine kinase inhibitor such as imatinib, nilotinib and dasatinib, and (ii) defensin α1 and/or defensin α2 and/or defensin α3 and/or defensin α4, and/or an agent triggering an increase of defensins in humans. The present invention also concerns a kit of parts comprising the same elements, in separate vials or packages.

Other characteristics of the invention will also become apparent in the course of the description which follows of the biological assays which have been performed in the framework of the invention and which provide it with the required experimental support, without limiting its scope.

### FIGURES LEGENDS

FIG. 1: BCR-ABL/ABL and defensin α1/β actin evolution in a secondary resistant patient.

FIG. 2: evolution of the expression ratios BCR-ABL/ABL and defensin α1/β actin in a patient who stopped imatinib therapy.

FIG. 3: comparison of defensin α1 expression at J0 imatinib in both responders and non-responders populations. Dashes indicate mean values

FIG. 4: Q-RT/PCR analysis of BCR-ABL and DEFA1 expression. BCR-ABL expression levels are presented as expression ratios compare to ABL. DEFA1 expression levels are presented as expression ratios compare to O-actin.

(A) Kinetic of BCR-ABL expression in a patient with secondary IM resistance. Arrows A and B indicate samples used for the microarray experiment.

(B) Example of kinetics of BCR-ABL and DEFA1 expression in two patients with secondary IM resistance. The levels of BCR-ABL mRNA transcripts are represented by continuous lines. The levels of the DEFA1 transcripts are illustrated by the discontinuous lines.

FIG. 5: DEF1A expression level in imatinib-responding (n=41) and imatinib-resistante (n=21) patients before imatinib treatment initiation.

FIG. 6: Examples of CML patient responses to imatinib daily dose increase. BCR-ABL/ABL ratios as shown in continuous lines and defensin α1/β actin gene expression ratios are shown in discontinuous lines.

(A) Imatinib dose increase leads to BCR-ABL/ABL gene expression decrease and to defensin α1/β actin gene expression decrease.

(B) Imatinib dose increase leads to BCR-ABL/ABL gene expression decrease and to defensin α1/β actin gene expression increase.

### EXAMPLES

The experimental data described in examples 1 to 10 below have been obtained by using the materials and methods which follow.

Patient Characteristics

A total of 44 patients were enrolled in this study. The mean age at diagnosis time of all patients was 52 years (range: 19-79), with 16 female and 28 male patients. The mean time between diagnosis and imatinib treatment was 21 months (range 0-87). Twenty-six patients received another treatment prior to imatinib therapy.

Peripheral blood samples used for this study were collected after informed consent was obtained in accordance with the Declaration of Helsinki.

Plasmids

Defα1, Defα4, βACT and HuP0 PCR fragments were obtained using DEFα1 forward primer 5′-AGGCTCAAGGAAAAACATGG-3′ (SEQ ID No: 1) and reverse primer 5′-GCAGAATGCCCAGAGTCTTC-3′ (SEQ ID No: 2), DEFα4 forward primer 5′-GCAGCTGAGCTTGCAGAATA-3′ (SEQ ID No: 3) and reverse primer 5′-GGACAAAGTATAGGAGAAACAACCA-3′ (SEQ ID No: 4), ACT forward primer 5′ AGCATCGGGTGATGTTCATT-3′ (SEQ ID No: 5) and reverse primer 5-ATTACAAGCATGCGTCACCA-3′ (SEQ ID No: 6) HuP0 forward primer 5′TGGAGGGTGTCCGCAATGTT-3′ (SEQ ID No: 7) and reverse primer 5′-GAAGGCCTTGACCTTTTCAG-3′ (SEQ ID No: 8) respectively. These fragments were inserted into the pCR®2.1-Topo vector using the TOPO TA Cloning® kit (Invitrogen). The resulting plasmids were used as template DNA at concentrations ranging from 10⁸ to 10¹ copies/μl to produce standard curves.

RQ-PCR

Total RNA from leucocytes was extracted using Trizol reagent method (Invitrogen). DNA contaminants were removed by DNase I treatment (Ambion). RNA concentration was determined by OD260. cDNA was synthesized from 1 μg of total RNA with random hexamers in a final volume of 20 μl (Roche). Quantitative polymerase chain reaction (RQ-PCR) was carried out in 96-well ABgene plates using the Mx3005P system (Stratagene) with the Sybr green Master mix reaction (Stratagene). All reactions were performed in a total volume of 25 μl and contained 2 μl of cDNA and 6.25 μM of each primer set. Each sample was analyzed in triplicate. Negative controls without added reverse transcriptase were performed. The primers used for amplification of DEFα1, DEFα4, ACT and HuP0 were the same as described above. Thermal cycling was performed at 95° C. for 10 min followed by 40 cycles comprising each a denaturation step at 95° C. for 30 s, and an annealing/extension step at 58° C. for 45 s. Amplification of the appropriate product was verified by continuous monitoring of the fluorescence; the temperature was ramped from 58° C. to 95° C. by steps of 0.1° C. to generate a melting curve.

BCR-ABL/ABL transcript levels were quantified as previously described (Colombat et al., 2006).

Microarray Experiments

Total RNA was purified on Rneasy Mini Kit column for removal of small fragments that affect RT-reaction and hybridization quality (Qiagen). Microarray used were the 22K Human Agilent Microarray. The targets for Agilent DNA microarray analysis were prepared according to the manufacter's instructions. The amount of starting total RNA for each reaction was 500 ng. Briefly, first strand cDNA synthesis was generated using a T7-linked oligo-dT primer, followed by second strand synthesis. An in vitro transcription reaction was performed to generate cRNA containing Cy3-CTP or Cy5-CTP. Labeling and hybridization of RNA for microarray analysis were performed using the Agilent low RNA input linear amplification kit. Microarray hybridizations were carried out on Agilent Human oligonucleotide microarrays using 0.75 μg Cy3-labeled “A” sample and 0.75 μg Cy5-labeled “B” sample. Hybridizations were carried out overnight using the Agilent hybridization kit and a “22K” chamber hybridization oven. The arrays were washed once in 6×SSC and 0.005% Triton X-102 10 min at room temperature and once in 0.1×SSC and 0.005% Triton X-102 5 min at 4° C. Microarrays were scanned using Dual Laser Microarray Scanner (Agilent Technologies).

Statistical Analysis

Statistical analysis was carried out using Mann-Whitney test, or Student's t-test for bigger cohorts (comprising more than 30 individuals).

### Example 1

### Identification of Defensin α1 and Defensin α4 as Potential Markers for Relapse of CML Patients Treated by Imatinib

The inventors have performed a transcriptomic study of the imatinib resistance in three patients exhibiting a secondary resistance. Differential RNA expression profiles were analysed using microarray. For each patient taken separately, gene expression of said patient when responding to treatment was compared to gene expression of the same patient during relapse. It was noted that the BCR-ABL levels were about the same in both conditions (respond and relapse). By doing so, the inventors could get rid of genetic variability, since each patient was his own control; moreover, the results were not due to the variability of BCR-ABL level. The median time between CML diagnosis and the imatinib treatment was 38 months. Agilent 22K human 60-mer oligonucleotide pan genomic microarrays (Agilent technologies) were used for this study. Four experiments (a dye-swap and a duplicate) were done for each patient. The selective criteria were drastic. A gene was selected only if the expression difference was positive in each experiment; moreover, the inventors selected only genes presenting a log ratio above 0.5 or below −0.5, with a p-value below 0.001. This corresponds at least to a three times variation expression.

With these criteria, genes coding for the defensin α1 and defensin α4 were highly over-expressed, about 50 times when patients relapsed.

Microarray results were then confirmed by quantitative reverse transcriptase polymerase chain reaction assay (RT-PCRq), which is more sensitive. With this technique, the over-expression was about 300 times when patients relapsed.

### Example 2

### Defensin α1 and Defensin α4 Levels in Primary Resistant Patients

The inventors then investigated defensin α1 and defensin α4 expressions in responder patients to the imatinib treatment and in patients primary resistant to the treatment at different periods of their disease. The reference gene used in the RT-PCR quantitative experiment disclosed in Examples 1 to 5 was the gene encoding the β actin. Other reference genes can be used alternatively. In particular, the gene coding the acidic ribosomal P0 protein (HuP0) could advantageously be used, since it is expressed at a higher level than the gene encoding β actin, and hence, its use would allow to perform the experiments on smaller samples. In what follows, the defensin level in hence calculated as: defensin α1 copy number/β-actin copy number.

In primary resistant patients, defensin levels before imatinib were about 10⁶ and 3 to 6 months later, defensin levels were about 10⁴. In responder patients, defensin levels were also about 10⁷-10⁶ and 3 to 9 months after imatinib therapy, defensin levels ranged between 0.9 and 129. In this later case, there was a strong decrease of the imatinib rate.

### Example 3

### Defensin α1 Level in Secondary Resistant Patients

From the first results disclosed in Examples 1 and 2, the inventors assumed that in secondary resistant patients the relapse was accompanied by an increase of defensin α1 and defensin α4 mRNA, whereas in primary resistant patient the defensin mRNA remained high and in responder patient there was a strong and persistent reduction of the defensin transcription level.

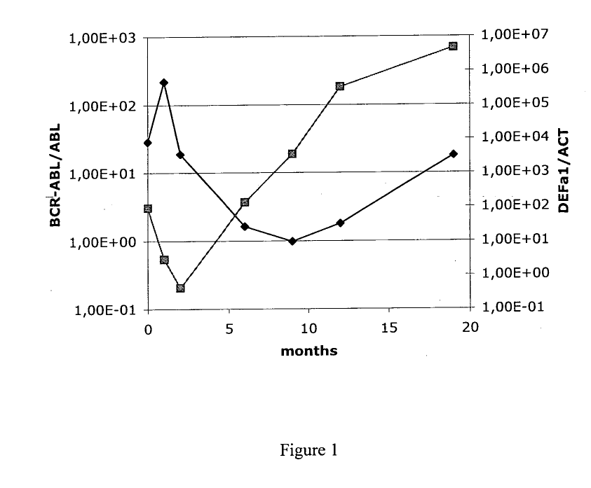

The inventors thus looked more specifically at secondary resistant patients. For these patients, BCR-ABL/ABL gene expression ratio and defensin α1/β actin gene expression ratio were measured at different times. Total RNA was extracted from leucocytes from a single blood tube and then treated with Dnase I to remove residual contamination of genomic DNA. RNA was transcribed in cDNA and a PCRq assay was realized to determine defensin α1/β actin and BCR-ABL/ABL copy numbers ratios. These results allow to quantify the expression level of defensin α1 and BCR-ABL/ABL. Example of BCR-ABL/ABL and defensin α1/β actin genes expression curves in a secondary resistant patient is shown in FIG. 1.

As appears from FIG. 1, defensin α1/β actin ratio increases 6 months before the BCR-ABL/ABL ratio.

To validate that the defensin α1 could be used as an early predictive marker of imatinib resistance, 8 secondary resistant patients were then studied. In all these cases, defensin α1 expression increased in average 6 months before BCR-ABL.

### Example 4

### Defensin α1 and Defensin α4 Levels in Patients in Remission

Patients in complete molecular remission are patients who have had repeated negative results by BCR-ABL/ABL real-time polymerase chain reaction during at least 2 years. The inventors investigated the defensin levels in some of these patients, who have stopped imatinib therapy.

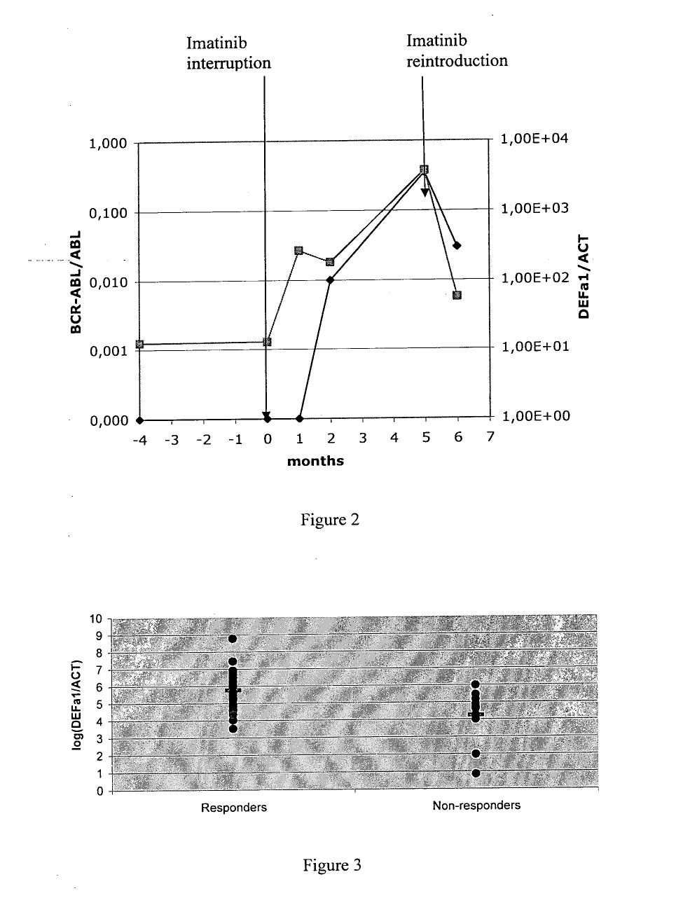

An example of BCR-ABL/ABL and defensin α1/β actin gene expression evolution over time in a patient who stopped imatinib therapy is shown in FIG. 2.

For this patient, the defensin α1/β actin gene expression ratio increased one month after the imatinib therapy stop, whereas the BCR-ABL/ABL gene expression ratio increased two months after the imatinib therapy stop.

Three other patients in complete molecular remission have been analyzed. One patient who stopped imatinib therapy two years ago did not relapse. For this patient, BCR-ABL is still undetectable and the defensin α1 expression is stable. The two other patients relapsed after they stopped imatinib therapy and the increase of defensin α1 expression occurred at the same time and 3 months before the increase of BCR-ABL/ABL gene expression.

These experiments reinforce the role of defensin α1 as an early predictive marker of CML relapse.

Similar results were obtained with defensin α4.

Defensin α1 and defensin α4 expression levels can hence be used as early predictive genetic markers of CML patient relapse.

Even with low BCR-ABL/ABL level, undetectable. Philadelphia chromosome and normal blood formula, it is now possible to predict relapse in CML patients. In this way, treatment can be changed rapidly, for example by increasing imatinib doses, using other tyrosine kinase inhibitors or performing a bone marrow transplantation.

### Example 5

### Defensin α1 and Defensin α4 Levels as Markers for Predicting a Patient's Response to Imatinib Treatment

Defensin α1 expression levels were measured at day 0 of imatinib therapy, in 28 responder patients and 11 non-responder patients (3 primary resistant and 8 secondary resistant patients). The results are given in FIG. 3.

In responder and non-responder populations, defensin α1 expression medians are 5.8 and 4.7 respectively and defensin α1 expression means are 5.8 and 4.2 respectively (wherein defensin α1 expression is calculated as the log of the ratio [defensin α1 copy number/β-actin copy number]). Mann-Whitney test was used to test for significant difference between defensin α1 expression and response of patients. Level of significance was set at 3%. The statistical power of the test is 84%. Statistical analysis showed significantly defensin α1 expression difference between responder versus non-responder population.

Similar results are obtained with defensin α4.

Defensin α1 and defensin α4 can hence be used also as predictive markers of imatinib treatment response.

The identification of defensin α1 or defensin α4 as early predictive genetic markers is essential for the treatment of CML.

### Example 6

### Defensin α2 and Defensin α3 can Also be Used for predicting the evolution of a patient suffering or having suffered from chronic myeloid leukemia

Defensin α1 and defensin α3 (also called HNP-1 and HNP-3, respectively) differ only by the first amino acid of the mature peptides, whereas defensin α2 (HNP-2) lacks this residue. Since no gene for defensin α2 has been discovered, it is thought that this peptide is a proteolytic product of one or both defensin a 1 and defensin α3. Aldred et al. have demonstrated that in people having both defensin α1 and defensin α3 genes (some people lack defensin α3 gene), the ratio between defensin α1 and defensin α3 mRNAs depends from the copy numbers of each gene, whereas there is no relationship between the total (defensin α1+defensin α3) mRNA levels and the total gene copy number (Aldred et al., 2005).

The data presented in examples 1 to 5 and in the following ones have been obtained with primers which cannot differentiate defensin a 1 and defensin α3 mRNAs. Hence, what has been measured is in fact the expression level of defensin al and defensin α3 genes.

The present invention can hence be performed by measuring:
the expression level (mRNAs levels) of defensin α1 and defensin α3.
the expression level of defensin α1 alone or defensin α3 alone (with specific primers, for example);
the level of peptides HNP-1, HNP-2 and HNP-3, either alone or in combination;
the expression level of defensin α4; and/or
the level of peptide HNP-4.

### Example 7

### Use of Defensins as Therapeutic Agents

Higher levels of defensin α1 found in imatinib good responder patients before the treatment highlights the possibility of a putative role of defensins in tumor suppression. Such a role was suggested by Bullard et al. for the defensin β1 in prostate cancer (Bullard et al., 2008). This tumor suppressor effect of defensin α1 (and of defensins α4, α2 and α3) could be necessary for the success of imatinib treatment. A further study is performed to determine if these defensins can be used as therapeutic or prophylactic agents (to prevent a relapse of CML) either alone or in combination with imatinib. In such a case, defensins can be administered either as such, or an agent inducing the secretion of defensins can be used instead, such as bacteria or fungi.

### Example 8

### Analysis of Defensin α1 and Defensin α4 Levels in a Larger Number of CML Patients in Chronic Phase (CML-CP)

Imatinib (IM) is the current standard of care in patients with chronic myeloid leukaemia (CML), inducing durable responses and prolonged progression-free survival (Druker et al., 2006). Despite a remarkable effectiveness, cases of treatment failure have been reported. Currently known baseline prognostic factors for response to IM include the phase of the disease at IM initiation and relative risk assessed by Sokal and Hashford scores as well as the so called “late chronic phase” CML that refers to patients who have been treated with others therapies before imatinib initiation (Druker et al., 2006). Since alternative therapeutic options are available, identification of additional predictive factors of IM resistance is of interest. In this study, the inventors focused on the identification of specific early predictive biomarkers of IM resistance.

To this aim, they performed pangenomic microarray analysis in patients with secondary IM resistance. Two genes encoding α-defensin 1 (DEFA1) and α-defensin 4 (DEFA4) were highly expressed at the time of IM resistance. Because α- and β-defensins had already been described as putative biomarkers in different cancers (Holterman et al., 2006; Nam et al., 2005), DEFA1 and DEFA4 expression was investigated in CML-CP patients responding and resistant to IM treatment. The obtained data suggest that variation of DEFA1 and DEFA4 expression is associated early on with IM resistance while a low level of DEFA1 and DEFA4 expression in CML patients before imatinib treatment initiation is a predictive factor for IM resistance.

All patients enrolled in this study provided informed consent according to the Declaration of Helsinki. CML-CP patients treated with IM standard dose (400 mg/day) were considered for this study. Primary resistance to IM was defined as lack of cytogenetic response after at least six months of IM therapy. Secondary resistance was defined as loss of previous, at least partial, cytogenetic response.

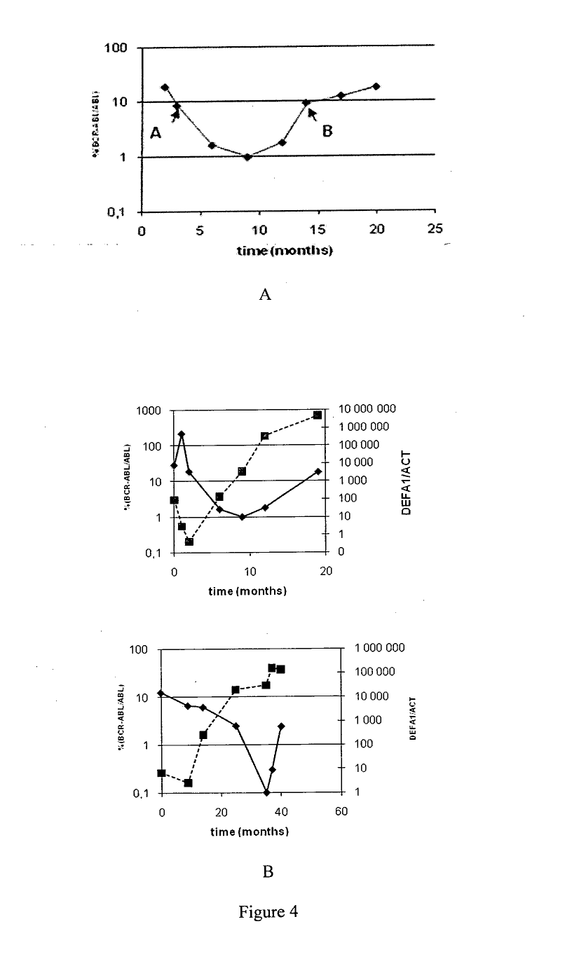

To search predictive biomarkers of IM resistance in CML, transcriptomic analysis was performed on a few patients. Four patients with secondary IM resistance, still in complete haematological response at the time of analysis, were selected for microarray analysis. Gene expression was compared separately for each patient when patients respond to treatment and when patients relapse (FIG. 4A). Moreover, the BCR-ABL mRNA transcript level and blood count were the same in both conditions (response and relapse). This avoids genetic variability as each patient is his/her own control; removing the possibility that the results were due to the variability of BCR-ABL expression or a different blood cell count. Eighteen down-regulated genes and 4 up-regulated genes that showed significant differential expression variation (variation higher than 0.5 log or less than −0.5 log with a p-value less than 0.001 in all samples) were selected. Among them, the DEFA1 and DEFA4 genes were highly over-expressed at the time of resistance to IM (63-fold and 30-fold, respectively). The over-expression of both genes was confirmed by Q-RT/PCR in the patients used in microarray experiments.

Since over-expression of DEFA1 seemed to be associated with secondary IM resistance, a prospective analysis of DEFA1 expression in CML-CP during IM therapy was performed. Samples from 9 responders and 6 secondary resistant patients were analyzed by Q-RT/PCR for DEFA1 expression. In all IM-responding patients, a long-lasting decrease of DEFA1 mRNA transcript level with a DEFA1 to β-actin ratio stabilized around 10³ is observed. In IM-resistant patients the DEFA1 expression decrease is followed by a dramatic increase of DEFA1 expression after a few months. Moreover, in IM-resistant patients, a lower DEFA1 expression before IM initiation is observed independently of the treatment prior imatinib received by the patient.

In addition, kinetics of BCR-ABL and DEFA1 expression was done on these patients. In IM-responding patients, BCR-ABL and DEFA1 expression decreases are parallel. In IM-resistant patients, DEFA1 expression began to increase when BCR-ABL mRNA transcript level was still decreasing. An example of kinetics of BCR-ABL and DEFA1 expression in two IM-resistant patients is shown in FIG. 4B. The median duration of time between the increase of DEFA1 expression and increase of BCR-ABL mRNA transcript level was 6 months (range from 0 to 23 months) suggesting that variation in the DEFA1 expression may not be correlated to that of BCR-ABL. DEFA1 is identified as candidate marker that may be useful for early identification of relapse in CML IM-patients.

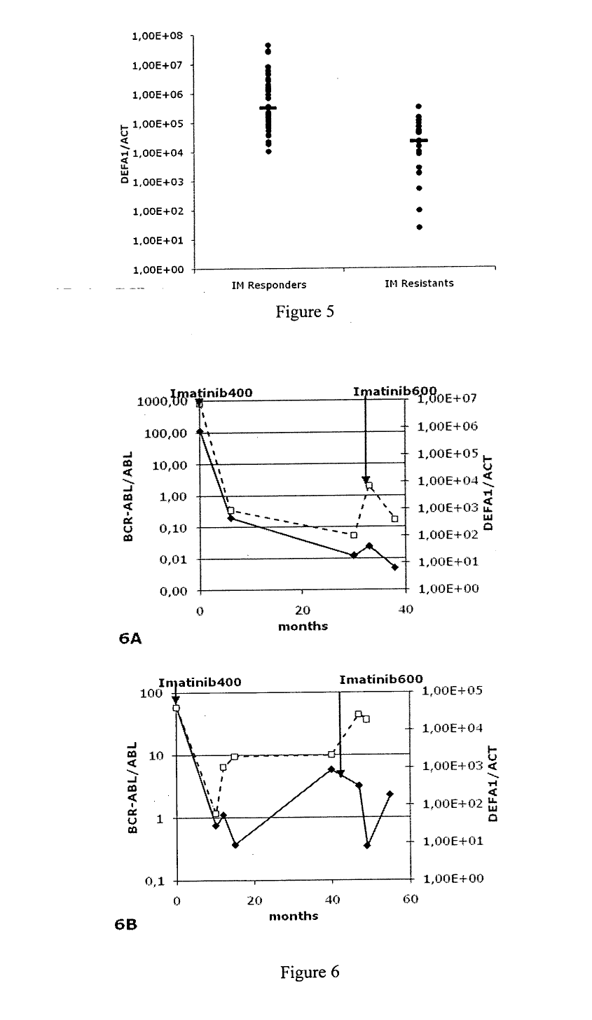

Finally, as variation of DEFA1 expression is associated with IM resistance, DEFA1 expression in IM-responding (n=41) and IM-resistant (n=21) patients before IM initiation was compared (FIG. 5). In responders and IM-resistant patients, DEFA1/β-actin expression means were 3.8×10⁶ and 5×10⁴ and medians were 3.3×10⁵ and 2.1×10⁴, respectively. Student's t-test showed significant difference between responders versus IM-resistant patients with a p value of 0.01. DEFA1 is also identified as candidate marker that may be useful for identification before IM initiation of IM-resistant CML patients.

All experiments were also performed for DEFA4 expression and similar results were obtained (results not shown).

Previous studies using transcriptomic analyses have pointed out variation of α-defensin expression in CML patients. Nowicki et al. reported down-regulated expression of DEFA1 and DEFA4 in CML-CP cells in comparison to their normal counterparts (Nowicki et al., 2003). Hagberg et al. assessed higher expression of DEFA4 prior to interferon alpha therapy in interferon alpha resistant patients but the difference between the two groups in DEFA4 expression was not found to be significant using Q-RT/PCR (Hagberg et al., 2007). Although the significance of such variations remains unclear, taken together with the results presented here these suggest a particular role of a-defensins in CML. Moreover, DEFA1 expression was investigated using Q-RT/PCR in various haematological disorders prior therapy, including acute myeloid leukaemia, myelodysplasia, Philadelphia-negative myeloproliferative syndromes, CML and healthy donors. Interestingly, DEFA1 expression was highest in CML patients with a mean DEFA1/β-actin ration of 10⁶ copies, whereas in other malignancies or healthy donors this was found to be 10³ or less (data not shown). Defensins are small peptides involved in innate and adaptative host defence mechanisms. Two classes of defensins have been identified, alpha and beta, that differ with respect to their structure and tissue pattern of expression. They are synthesized as prodefensins in the bone marrow precursors of blood granulocytes and mature defensins are stored in the granules of neutrophils. Numerous studies have pointed out their crucial role in linking innate immunity with the adaptative immune system (Yang et al., 2002). As variation of DEFA1 expression appeared not to be correlated with that of BCR-ABL, we hypothesized that the relatively high expression of DEFA1 may reflect an adequate immune control of the disease allowing long-lasting IM response. A putative role of defensin in tumor suppression was highlighted by Bullard et al. about the defensin β1 in prostate cancer (Bullard et al., 2008). This putative tumor suppressor effect of defensin a 1 could be necessary for the imatinib treatment. It can be speculated that the low expression of defensin interferes with immune system and contributes to the non response.

In conclusion, the inventors have demonstrated that quantification of DEFA1 expression is of interest in order to early identify IM-resistant patients, as new therapeutic options are available.

### Example 9

### Defensin α1 and Defensin α4 Levels as Markers for Predicting a Patient's Response to an Increase of Imatinib Daily Dose

Forteen (14) patients who did not respond well to imatinib treatment at the usual dose of 400 mg/day were studied. For all of them, a dose increase to imatinib 600 (600 mg/day) was performed. Recently, Picard et al. have shown that imatinib plasma level was around 1000 ng/ml in treated patients (Picard et al., 2007). In case of lower concentration, it is recommended to increase the imatinib dose.

In fact, imatinib plasma level is not systematically realized. When patient response to imatinib treatment is not good enough, the physician usually increases imatinib dose. To analyze the effect of the dose increase on patients, BCR-ABL/ABL kinetic is performed. In major cases, dose increase leads to a decrease of BCR-ABL/ABL expression. But in some cases, this decrease lasts only a few months, showing that imatinib dose increase is not the good therapy to decrease BCR-ABL/ABL expression in a long lasting way.

To determine quickly dose increase effect, it is interesting to follow at the same time BCR-ABL/ABL expression and defensin α1 gene expression (as a ratio with β actin gene expression, for example). Indeed, if imatinib dose increase is necessary and sufficient to improve patient response, defensin α1 gene expression and BCR-ABL/ABL expression will decrease at the same time (FIG. 6A). If imatinib dose increase is not sufficient to lead to a BCR-ABL/ABL expression decrease, defensin α1 gene expression will increase while BCR-ABL/ABL expression will transiently decrease (FIG. 6B)

These results show that defensin α1 gene expression can be used as a molecular marker to estimate the effect of imatinib dose increase in CML patients treated with imatinib.

### Example 10

### Effects on Defensin α1 and Defensin α4 Levels of Treatment by Tyrosine Kinase Inhibitors Different from Imatinib

The same experiments as described above have been performed with second generation tyrosine kinase inhibitors, such as Nilotinib (Novartis) and Dasatinib (BMS). The same results as described above have been observed. The above conclusions and applications can hence be extended to any tyrosine kinase inhibitor.

### REFERENCES

Aldred, P. M., Hollox, E. J., and Armour, J. A. (2005). Copy number polymorphism and expression level variation of the human alpha-defensin genes DEFA1 and DEFA3. Hum Mol Genet 14, 2045-2052.
Bullard, R. S., Gibson, W., Bose, S. K., Belgrave, J. K., Eaddy, A. C., Wright, C. J., Hazen-Martin, D. J., Lage, J. M., Keane, T. E., Ganz, T. A., and Donald, C. D. (2008). Functional analysis of the host defense peptide Human Beta Defensin-1: new insight into its potential role in cancer. Mol Immunol 45, 839-848.
Colombat, M., Fort, M. P., Chollet, C., Marit, G., Roche, C., Preudhomme, C., Reiffers, J., Praloran, V., and Mahon, F. X. (2006). Molecular remission in chronic myeloid leukemia patients with sustained complete cytogenetic remission after imatinib mesylate treatment. Haematologica 91, 162-168.
Deininger, M., Buchdunger, E., and Druker, B. J. (2005). The development of imatinib as a therapeutic agent for chronic myeloid leukemia. Blood 105, 2640-2653.
Druker, B. J., Guilhot, F., O'Brien, S. G., Gathmann, I., Kantarjian, H., Gattermann, N., Deininger, M. W., Silver, R. T., Goldman, J. M., Stone, R. M., et al. (2006). Five-year follow-up of patients receiving imatinib for chronic myeloid leukemia. N Engl J Med 355, 2408-2417.
Faderl, S., Talpaz, M., Estrov, Z., O'Brien, S., Kurzrock, R., and Kantarjian, H. M. (1999). The biology of chronic myeloid leukemia. N Engl J Med 341, 164-172.
Hagberg, A., Olsson-Stromberg, U., Wickenberg-Bolin, U., Goransson, H., Isaksson, A., Bengtsson, M., Hoglund, M., Simonsson, B., and Barbany, G. (2007). Gene expression analysis identifies a genetic signature potentially associated with response to alpha-IFN in chronic phase CML patients. Leuk Res 31, 931-938.
Holterman, D. A., Diaz, J. I., Blackmore, P. F., Davis, J. W., Schellhammer, P. F., Corica, A., Semmes, O. J., and Vlahou, A. (2006). Overexpression of alpha-defensin is associated with bladder cancer invasiveness. Urol Oncol 24, 97-108.
Nam, M. J., Kee, M. K., Kuick, R., and Hanash, S. M. (2005). Identification of defensin alpha6 as a potential biomarker in colon adenocarcinoma. J Biol Chem 280, 8260-8265.
Nowicki, M. O., Pawlowski, P., Fischer, T., Hess, G., Pawlowski, T., and Skorski, T. (2003). Chronic myelogenous leukemia molecular signature. Oncogene 22, 3952-3963.
Picard, S., Titier, K., Etienne, G., Teilhet, E., Ducint, D., Bernard, M. A., Lassalle, R., Marit, G., Reiffers, J., Begaud, B., et al. (2007). Trough imatinib plasma levels are associated with both cytogenetic and molecular responses to standard-dose imatinib in chronic myeloid leukemia. Blood 109, 3496-3499.
Yang, D., Biragyn, A., Kwak, L. W., and Oppenheim, J. J. (2002). Mammalian defensins in immunity: more than just microbicidal. Trends Immunol 23, 291-296.

1. A method for predicting the response to a patient suffering from chronic myeloid leukemia to treatment with a tyrosine kinase inhibitor comprising detecting the presence in the patient of a defensin selected in the group consisting of defensin α1, defensin α2, defensin α3, and defensin α4, or of a combination thereof wherein the presence of the defensin indicates susceptibility to the tyrosine kinase inhibitor.

2. The method of claim 1, wherein said tyrosine kinase inhibitor is imatinib, nilotinib or dasatinib.

3. A method for predicting relapse in a patient who is treated or who has been treated for a chronic myeloid leukaemia, wherein said method comprises the following steps:
(a) in vitro measuring the expression level of defensin α1 and/or defensin α4;
(b) comparing the measured expression level of defensin α1 and/or defensin α4 to a predetermined threshold;
wherein a measured level above said predetermined threshold is indicative of relapse.

4. The method of claim 3, wherein said predetermined threshold is calculated as 10×P, wherein P is the level of the same marker previously measured in the same patient, when said patient's condition was improving or stabilized.

5. The method of claim 3, wherein said predetermined threshold is 10⁴-fold the expression level of β-actin.

6. A method for following the evolution of a patient who has been treated for a chronic myeloid leukaemia, wherein said method comprises the following steps:
(a) in vitro measuring the expression level of defensin α1 and/or defensin α4 in biological samples from said patient, wherein said biological samples have been obtained at various dates;
(b) comparing the measured expression levels of defensin α1 and/or defensin α4 in said biological samples;
wherein an increase in the level of expression of defensin α1 and/or defensin α4 in said patient is indicative of relapse.

7. A method for determining if a patient suffering from chronic myeloid leukaemia is likely to be a good responder to a treatment with a tyrosine kinase inhibitor, comprising the following steps:
(a) in vitro measuring the expression level of defensin α1 and/or defensin α4;
(b) comparing the measured expression level of defensin α1 and/or defensin α4 to a predetermined threshold;
wherein a measured level above said predetermined threshold indicates that said patient is likely to be a good responder to the treatment.

8. A method for predicting if a patient suffering from chronic myeloid leukaemia and for whom a treatment with a tyrosine kinase inhibitor is insufficient can benefit from an increase of daily dose of said tyrosine kinase inhibitor, comprising the following steps:
(a) in vitro measuring the expression level of defensin α1 and/or defensin α4 prior to and after the dose increase;
(b) comparing the measured expression levels of defensin α1 and/or defensin α4;
wherein a decrease of α1 and/or defensin α4 expression level following the dose increase is indicative of a good response, whereas an increase of said expression level is indicative of treatment failure.

9. The method of claim 7, wherein said inhibitor is imatinib, nilotinib or dasatinib.

10. The method of claim 3, wherein the expression level of defensin α1 is measured.

11. The method of claim 3, wherein the expression level of defensin α4 is measured.

12. The method of claim 3, wherein the expression levels of both defensin α1 and defensin α4 are measured.

13. The method of claim 3, wherein the expression level of defensin α1 and/or defensin α4 is measured by quantitative polymerase chain reaction.

14. The method of claim 3, wherein the expression level of defensin α1 and/or defensin α4 is calculated as a ratio between the copy number of mRNA encoding said defensin and the copy number of mRNA encoding a reference gene.

15. A pharmaceutical composition comprising at least a tyrosine kinase inhibitor and an agent selected in the group consisting of defensin α1, defensin α2, defensin α3, defensin α4 and agents inducing an increase of defensin α1 and/or defensin α2 and/or defensin α3 and/or defensin α4 when administered to a human.

16. A kit of parts comprising at least a tyrosine kinase inhibitor and an agent selected in the group consisting of defensin α1, defensin α2, defensin α3, defensin α4 and agents inducing an increase of the expression of at least one of said defensins when administered to a human.

17. A method for treating or preventing relapse of chronic myeloid leukemia in a human in need thereof comprising adminstering a composition comprising an effective amount of at least an agent selected in the group consisting of defensin α1, defensin α2, defensin α3, and defensin α4 and agents inducing an increase of the expression of at least one of said defensins when administered to a human.

18. The method of claim 16, wherein said composition is used in combination with a tyrosine kinase inhibitor.

19. A method of increasing the efficacy of a tyrosine kinase inhibitor in a human receiving treatment of chronic myeloid leukemia comprising administering a composition comprising an effective amount of an agent selected in the group consisting of defensin α1, defensin α2, defensin α3, defensin α4 and agents inducing an increase of the expression of at least one of said defensins when administered to a human.

20. The pharmaceutical composition of claim 15, the kit of claim 16, or the method of claim 18 or claim 19, wherein said tyrosine kinase inhibitor is imatinib, nilotinib or dasatinib.
